(19) United States
(12) Patent Application Publication
(10) Pub. No.: US 2004/0152694 A1
(43) Pub. Date: Aug. 5, 2004

(54) Methods and compositions for treating inflammatory disorders of the airways

(76) Inventors: Istvan Kurucz, Budapest (HU); Sandor Solyom, Budapest (HU); Viola Csillik nee Perczel, Budapest (HU)

Correspondence Address: Simona A. Levi-Minzi, Ph.D., 4400 Biscayne Boulevard, Miami, FL, 33137, US

(21) Appl. No.: 10/358,061

(22) Filed: Feb. 4, 2003

(51) Int. Cl. A61K 31/5513; A61K 31/517

(52) U.S. Cl. 514/221; 514/266.21

(57) ABSTRACT

The present invention provides compositions and methods for treating inflammatory disorders of the airways by the administration of a therapeutically effective amount of a modulator according to the invention. More specifically, the invention relates to the treatment of airway inflammations including asthma or an asthma-related pathologies.

### BACKGROUND OF THE INVENTION

1. Field of Invention

The invention relates to pharmaceutical compositions, and methods for preventing, treating or alleviating the symptoms of acute and chronic inflammatory disorders of the airways including allergic inflammations, such as asthma and asthma-related pathologies.

2. Summary of the Related Art

Inflammation is a multi-step cascade process, any part of which may be the subject of potential therapeutic intervention. Briefly, inflammation entails the infiltration of immunologically competent cells (for example eosinophils, mast cells, activated T-lymphocytes) into the injury site where they, together with resident cells, release bioactive mediator substances (eg, histamine, proteases, a host of cytokines and chemokines), which increase the permeability of nearby blood vessels, attract and stimulate bystander cells. The altered permeability of vessels results in a fluid exudate forming at the injury site followed by a further influx of reactive leukocytes and their eventual efflux into the damaged area. (For an overview see, Trowbridge and Emling, “Inflammation: A Review of the Process” Quintessence Pub. Co., 1997). Secretion of collagen and mucus by, and proliferation of, resident cells (smooth muscle and epithelial cells or fibroblasts stimulated by the released mediators) establish the extension of pathological alterations (eg., airway obstruction) and contribute to their development.

Inflammation is associated with a variety of pulmonary conditions including eg., intrinsic or extrinsic asthma bronchiale, any inflammatory lung disease, acute or chronic bronchitis, pulmonary inflammatory reactions secondary to chronic bronchitis, chronic obstructive lung disease, pulmonary fibrosis, as well as any pulmonary condition in which white blood cells may play a role including, but not limited to, idiopathic pulmonary fibrosis and any other autoimmune lung disease. Asthma is perhaps one of the most common forms of pulmonary inflammation affecting the large and small airways of the lung. It impacts on 5% to 10% of the human population, resulting in an estimated 27 million patient visits, 6 million lost workdays, and 90.5 million days of restricted activity per year. The morbidity and mortality rates for asthma are growing worldwide (Plaut and Zimmerman, “Allergy and Mechanisms of Hypetsensitivity” in Fundamental Immunology, 3^{rd} Ed., Paul (ed.), Raven Press, New York N.Y., at 1399 (1993)).

Conventional anti-asthma treatments have been predicated on the strict avoidance of all triggering allergens, which is inherently difficult to achieve, and on therapeutic regimens based on pharmacological agents having unfortunate side effects and suboptimal pharmacokinetic properties. β₂-adrenergic agonists used to treat bronchospasm have no effect on airway inflammation or bronchial hyperreactivity (Palmer et al, New Engl. J. Med. 331:1314 (1994)). Also, regular or prolonged use of β₂-adrenergic agonists is associated with poor control of asthma, increase in airway hyperresponsiveness to allergen, and reduced bronchoconstriction protection (Bhagat et al, Chest 108:1235 (1995)). Moreover, chronic use of β₂-adrenergic agents alone, by causing down regulation of β₂-adrenergic receptors, is suspected to worsen bronchial hyperreactivity. Theophyiline (an anti-asthma methylxanthine) is characterized by substantial variability in its absorbance and clearance. Corticosteroids, while relatively safe in adult patients, are toxic for children, resulting in adrenal suppression and reduced bone density and growth (Woolock et al, am. Respir. Crit. Care Med. 153:1481 (1996)). Cromolyn, used to prevent asthmatic episodes, is effective in preventing an asthmatic reaction only if given prior to an attack (Volcheck et al, Postgrad. Med. 104(3):127 (1998)). Antihistamines occasionally prevent or abort allergic asthmatic episodes, particularly in children, but often are only partially effective because of histamines are only one of many inflammation associated mediators (Cuss, “The Pharmacology of Antiasthma Medications”, in Asthma as an Inflammatory Disease, O'Byrne, Ed., Dekker, Inc., New York, at 199 (1990)) and O'Byne, “Airway Inflammation and Asthma”, in Asthma as an Inflammatory Disease, O'Byme, Ed., Dekker, Inc., New York, N.Y., 143 (1990)).

Thus, the current drug modalities suffer from a number of drawbacks. In general, the conventional agents have a relatively short duration of action and may be partially or wholly ineffective when administered after antigen challenge occurs. Moreover, because of serious adverse effects associated with the use of agents such as β2-adrenergic agonists and corticosteroids, the therapeutic margin of safety with such agents is relatively narrow and patients using them must be carefully monitored (see eg, WO 94/06783, WO 99/06025, U.S. Pat. Nos. 5,690,910 and 5,980,865). In a recent clinical study, with inhaled corticosteroids, there was only transient improvement in the airways function of 5-11-year-old asthmatic children after the first year of therapy, with regression to that observed with placebo over the next 3 years (The Childhood Asthma Management Program Research Group, N. Engl. J. Med., 343:1054 (2000)). This observation can best be explained by remodeling changes (characteristic feature of asthma) occurring in the airways that are refractory to corticosteroids (Davies, Curr. Opin. Allergy Clin Immol., 1:67 (2001)).

Glutamate receptors have lately been found in cells not belonging to the central nervous system (Skerry and Genever: Trends in Pharmacol. Sci., 22:174 (2001)) and NMDA-receptors have recently been identified in the lung (Said et al, Proc. Natl. Acd Sci. (USA) 93:4688 (1996)). Activation of the NMDA receptor here triggers an acute edematous lung injury similar to that observed in “acute respiratory distress syndrome” (Said et al., Proc. Natl. Acad. Sci. USA 93:4688 (1996)). It has also been suggested that inflammatory injury may result from the activation of the NMDA receptor of asthmatic airways (Said, Trends in Pharmacol. Sci. 20:132 (1999)). Glutamate antagonists have also shown activity outside of the neurological arena. Rzeski et al (Proc. Natl. Acad. Sci. USA, 98:6372 (2001)) have shown in vitro anti-proliferative activity of both AMPA and NMDA antagonists against a panel of tumor cell types.

Much interest has been generated in identifying moieties capable of inhibiting the activation of the AMPA receptors, thereby leading to the identification of both competitive and non-competitive AMPA receptor antagonists. AMPA receptor modulators are currently under investigation for their potential utility as therapeutic agents for numerous central nervous system disorders including ischemia, epilepsy, schizophrenia, memory impairment, and drug abuse (see, eg., Lees, Drugs, 59:33 (2000) for a review).

AMPA receptor antagonists are referred to in several published patents and patent applications including the following issued U.S. Pat. Nos. 4,812,458; 5,192,792; 5,525,584; 5,268,378; 5,270,306; 5,342,946; 5,356,902; 5,364,876; 5,376,784; 5,395,827; 5,399,696; 5,407,935; 5,420,155; 5,426,106; 5,446,051; 5,475,008; 5,504,085; 5,510,338; 5,631,373; 5,514,680; 5,519,019; 5,521,174; 5,527,810; 5,532,236; 5,559,106; 5,559,125; 5,580,877; 5,606,062; 5,614,508; 5,614,532; 5,672,705; 5,721,234; 5,639,751; 5,654,303; 5,807,851; 6,060,479; 6,075,018; 6,096,743; and 6,200,999, published international (PCT) patent applications: WO 92/07847; 93/05772; 94/25469; 95/311443; 95/35289; 96/11922; 96/14318; 96/28445; 96/37500; 97/10246; 97/19066; 97/25326; 97/25327; 97/25329; 97/28163; 97/34878; 99/07708 and published European patent applications: EP 0 315 959 A2; 0 348 872 A1; 0 374 534 A1; 0 377 112 A1; 0 459 561 A2; 0 503 349 A1; 0 511 152 A2; 0 676 397 A1; and 0 699 676 A1.

GYKI 52466 (1-(4-aminophenyl)-4-methyl-7,8-methylenedioxy-5H-2,3-benzodiazepine) is a non-competitive AMPA-antagonist compound disclosed in U.S. Pat. No. 4,614,740. GYKI 52466 has been shown to be a highly selective antagonist of AMPA receptors, with a non-competitive mode of action, ie, it acts at an allosteric site (Donevan and Rogawski, Neuron 10:51 (1993); ibid. 10:61 (1993)). The non-competitive AMPA-antagonist effect of GYKI 52466 as well as its anticonvulsive, muscle-relaxant, and neuroprotective properties are well established (Upton, Trends in Pharmacol. Sci., 15:456 (1994)) by detailed pharmacological studies. Similarly the non-competitive antagonist LY 300164, a [(−)-1-(4-aminophenyl)-3-acetyl-4-methyl-7,8-methylenedioxi-3,4-dihydro-5H-2,3-benzodiazepine (GYKI-53773, also identified as Talampanel), has been shown to increase the anticonvulsant effects of diazepam and clonazepam and other antiepileptics (Borowitz et al, Eur. J. Pharmacol., 380:67 (1999), Czuczwar et al, Metab. Brain Dis., 11:143 (1996)). The potency of the protective effect observed varied with the epileptic model tested. Additionally, Talampanel was also shown to improve levodopa-induced dyskinesia in MPTP monkeys (Konitsiotis et al., Neurology 54:1589 (2000)).

European Patent Application No. 0548 370 A1 discloses a 2,3 benzodiazepine compound (1-(3,4-dimethoxyphenyl)-5-ethyl-7,8-dimethoxy-4-methyl-5H-2,3-benzodiazepine) also identified as Tofizopam which inhibits IgE production. Tofizopam has been postulated to bind to opioid receptors displaying anxiolytic potential (Horváth, Pharmazie 56(1):S56 (2001)). Tofizopam has not been shown to modulate the AMPA receptor to date.

So far, AMPA receptor associated effects upon local administration of antagonists and/or modulators or the physical presence of AMPA receptors in the respiratory system have not yet been demonstrated in the literature.

Therefore, there remains a need to identify and develop improved compositions and methods for treating inflammatory disorders of the airways, particularly allergic inflammations such as asthma and asthma-related pathologies. The identification and development of improved compositions and methods for the treatment of airway inflammatory diseases is especially important in the light of the limitations of the current strategies used for their causal management. In the case of asthma, the most prevalent inflammatory disease of the airways, currently available treatments focus almost exclusively either to suppress airway inflammation or to dilate obstructed airways, while other characteristic features of human asthma such as airway hyperreactivity and structural changes (eg., such as airways smooth muscle and fibroblast hypertrophy and/or hyperplasia, the accumulation of collagen and other matrix components in the subepithelial region of the airway wall, and the increased vascularity of the mucosa, collectively referred to as airway remodeling or destruction) (Kumar, Pharmacol. Therapeut., 91:93 (2001)), are largely ignored in the monitoring of the effectiveness of treatments.

### SUMMARY OF THE INVENTION

The present invention provides compositions and methods for the treatment of the inflammatory disorders of the airways. More specifically, the invention relates to the treatment of allergic airway inflammations including asthma or an asthma-related pathology. Diseases of an asthma-related pathology can include allergic reactions or an inflammatory disease of the airways that is no longer amenable to conventional treatment modalities, including airway remodeling.

The invention thus, provides methods for treating inflammatory disorders of the airways in a mammal by administering a therapeutically effective amount of an AMPA receptor modulator compound. In some embodiments of this aspect of the invention, the AMPA receptor modulator is either a non-competitive or a competitive AMPA receptor antagonist. The methods of this aspect of the invention are suitable for the treatment of allergic inflammatory disorders of the airways. Non-limiting representative inflammatory disorders of the airways contemplated include asthma, intrinsic or extrinsic asthma bronchiale, acute chronic bronchitis, pulmonary inflammatory reactions secondary to chronic bronchitis, chronic obstructive lung disease, and pulmonary fibrosis. Treatments according to this aspect of the invention are also suitable for other non-allergic inflammatory disorders of the airways including, but not limited to idiopathic pulmonary fibrosis and autoimmune lung disease.

Another aspect of the invention provides pharmaceutical compositions for the treatment of the inflammatory disorders of the airways in a mammal comprising a therapeutically effective amount of an AMPA receptor modulator compound in a pharmaceutically acceptable vehicle. In preferred embodiments of this aspect of the invention, the AMPA receptor modulator is either a non-competitive or a competitive AMPA receptor antagonist. Pharmaceutical compositions suitable for various modes of administration are disclosed.

### DETAILED DESCRIPTION

Surprisingly it has been found that compounds known to modulate the AMPA receptor are useful in treating inflammations of the airways. These AMPA receptor modulators have been found to reduce the bronchial airway hyper-responsiveness commonly associated with inflammations of the airways. Such compounds of the invention are also useful to suppress growth factor-induced proliferation of smooth muscle cells and inhibit allergen-induced mucus-secretion of airway epithelial cells thereby providing novel modalities for the treatment of airway obstruction. These findings have led to effective and versatile methods to treat inflammations of the airways and associated pathological states.

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

The present invention provides pharmaceutical compositions and methods for treating the symptoms of inflammatory disorders of the airways in a mammal. More specifically, the invention provides compositions and methods for the treatment of allergic airway inflammations, including asthma or an asthma-related pathologies, such as an allergic reaction or an inflammatory disease, which are no longer amenable to conventional therapies including for example airways remodeling.

The compositions and methods according to the invention also provide tools to investigate, in different experimental models, the role of compounds known to have AMPA receptor modulator properties, in the etiology and progression of various airway inflammations, including for example allergic airway inflammations, such as asthma and asthma-related pathologies, and airways remodeling.

Abbreviations:

AMOA, 2-Amino-3-[(3-carboxymethoxy)-5-methylisoxazol-4-yl]propionate

AMPA, α-Amino-3-hydroxy-5-methyl-4-isoxazole propionate

AMP 397A, 5-[N-(Phosphono-methyl)amino-methyl]-7-nitro-quinoxaline-2,3-dione

BIIR 561 CL (Irampanel), N,N-dimethyl-2-[2-(3-phenyl-1,2,4-oxadiazol-5-yl) phenoxy]ethanamine

Clonazepam, 7-Nitro-5-(2-chlorophenyl)-3H-1,4-benzodiazepin-2(1H)-one

CNQX, 6-Cyano-7-nitroquinoxaline-2,3-dione

CP 465,022, (S)-3-(2-chlorophenyl)-2-[2-(6-diethylaminomethyl-pyridin-2-yl)-vinyl]-6-fluoro-3H-quinazoline-4-one

CP-526,427, 3-(2-Chlorophenyl)-2-[2-(3-cyano-pyridin-2-yl)-vinyl]-6-fluoro-3-quinazoline-4-one

Diazepam, 7-Chloro-1,3-dihydro-1-methyl-5-phenyl-2H-1,4-benzodiazepin-2-one

DNQX, 6,7-Dinitroquinoxaline-2,3-dione

EGIS 8332, 5-(4-Aminophenyl)-7-acetyl-7,8-dihydro-8-cyano-8-methyl-9H-1,3-dioxo[4,5-h][2,3]benzodiazepine

GYKI 52466, 1-(4-Aminophenyl)-4-methyl-7,8-methylenedioxy-5H-2,3-benzodiazepine

GYKI 53405, 1-(4-Aminophenyl)-3-acetyl-4-methyl-3,4-dihydro-7,8-methylenedioxy-5H-2,3-benzodiazepine

GYKI 53655, 1-(4-Aminophenyl)-3-methylcarbamoyl-4-methyl-7,8-methylenedioxy-5H-2,3-benzodiazepine

GYKI 53773, (Talampanel, LY 300164), (−)-1-(4-Aminophenyl)-3-acetyl-4-methyl-3,4-dihydro-7,8-methylenedioxy-5H-2,3-benzodiazepine

GYKI 53784, (LY 303070), (−)-1-(4-Aminophenyl)-3-methylcarbamoyl-4-methyl-7,8-methylenedioxy-3,4-dihydro-5H-2,3-benzodiazepine

GYKI 47261, 1-(4-Aminophenyl)-8-chloro-2-methyl-11H-imidazo[1,2-c][2,3]-benzodiazepine

Indenone 4f, 9-Carboxymethyl-4,5-dihydro-4-oxo-imidazo[1,2-a]indeno[1,2-e]pyrazin-2-carboxylic acid

LU 112 313, 1-Carboxymethyl-7-(3-carboxy-pyrrol-1-yl)-6-nitroquinoxaline-2,3-(1H,4H)-dione

LU 115 455, N-((1-(1-Carboxymethyl-5,6,7,8-tetrahydro-benzo(f)quinoxaline-2,3-(1,4″-dion-9-yl)-pyrrol-3-yl)methyl-N′-(4-carboxyphenyl)-urea

LY 293 558, (LY 326 325), (3S, 4aR, 6R, 8aR)-decahydro-6-[2-(1H-tetrazol-5-yl)ethyl]-3-isoquinoline-3-carboxylic acid

MPTP, 1-Methyl-4-phenyl-1,2,3,6-tetrahydropyridine

NBQX, 2,3-Dihydroxy-6-nitro-7-sulphamoyi-benzo(f)quinoxaline

NS 257, 1,2,3,6,7,8-Hexahydro-3-(hydroxyumio)-N,N,7-trimethyl-2-oxo-benzo[2,1-b:3,4-c]dipyrrole-5-sulphonamide

NS 1209. (SPD 502), 8-Methyl-5-[4-(dimethylsulfamoy4)phenyl]-6,7,8,9-tetrahydro-1H-pyrrolo[3,2-h]isoquinoline-2,3-dione-3-O-(4-hydroxybutiric acid) oxime PNQX. (PD 152 247), 1,4,7,8,9,10-Hexahydro-9-methyl-6-nitropyrido[3,4-f]quinoxaline-2,3-dione

S-17625, [6,7-Dichloro-2(1H)oxoquinoline-3-yl]phosphonic acid

SYM 2207, (SYM 2189), 4-(Aminophenyl)-1-methyl-6,7-(methylenedioxy)-N-butyl-1,2-dihydrophthalazine-2-carboxamide

SYM 2267, 1-(4-Aminophenyl)-3,5-dihydro-4-methy-4-3-acetyl-7-methoxy-5H-2,3-benzodiazepine

TQX-173, 7-Chloro-4,5-dihydro-8-(1,2,4-triazol-4-yl)-4-oxo-1,2,4-triazolo[1,5-a]quinoxaline-2-carboxylic acid

YM 90K, (YM 900), 1,4-Dihydro-6-(1H-imidazol-1-yl)-7-nitro-2,3-quinoxalinedione

YM 872, (Zonampanel) 1,4-Dihydro-4-carboxymethyl-6-(1H-imidazol-1-yl)-7-nitro-2,3-quinoxaline-dione

ZK 200 775, (Fanampanel, MPQX), [[3,4-Dihydro-7-(4-morpholinyi)-2,3-dioxo-6-(trifluoromethy)-1 (2″)-quinoxalinyl]methy-4]phosphonic acid

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference is made in the following description and examples are obtainable from commercial sources, unless otherwise noted.

As used in this specification, the singular forms “a”, “an” and “the” specifically also encompass the plural forms of the terms to which they refer, unless the content clearly dictates otherwise. For example, reference to “a modulator” includes mixtures of modulators.

As used in this specification, whether in a transitional phrase or in the body of the claim, the terms “comprise(s)” and “comprising” are to be interpreted as having an open-ended meaning. That is, the terms ate to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, unless specifically indicated otherwise, the word “or” is used in the “inclusive” sense of “and/or” and not the “exclusive” sense of “either/or.”

The inventors have uncovered the presence of AMPA receptors in lung and have pioneered their role in pulmonary conditions heretofore unknown. The invention thus provides methods for treating inflammatory disorders of the airways in a mammal by administering a therapeutically effective amount of an AMPA receptor modulator.

The invention thus provides methods for treating inflammatory disorders of the airways in a mammal by administering a therapeutically effective amount of an AMPA receptor modulator.

As used herein, the terms “treating” or “treatment” are used to indicate reducing, alleviating, preventing and/or reversing the symptoms of a condition. Conditions to be treated by the methods and compositions of the invention include any condition characterized by, or including, acute and chronic inflaummtory disorders of the airways. Hence, the terms “inflammatory disorder” or “inflammatory disorders of the airways” encompass any inflammatory lung disease, including asthma, intrinsic or extrinsic asthma bronchiale, acute chronic bronchitis, allergic rhinitis, pulmonary inflammatory and structural reactions secondary to chronic bronchitis, chronic obstructive lung disease, pulmonary fibrosis. The invention is also useful for any pulmonary condition in which white blood cells and airway remodeling may play a role including but not limited to idiopathic pulmonary fibrosis and any other autoimmune lung disease.

By “asthma” is meant a condition of allergic origins, the symptoms of which include continuous or paroxysmal labored breathing accompanied by wheezing, a sense of constriction in the chest, and often attacks of coughing or gasping. By “asthma-related pathology” is meant a condition whose symptoms are predominantly inflammatory in nature with associated bronchospasm. Hence, both asthma and asthma-related pathologies are characterized by symptoms that include narrowing of airways, due in varying degrees to contraction (spasm) of smooth muscle, edema of the mucosa, including that of the upper airways and mucus in the lumen of the bronchi and bronchioles. Non-limiting representative examples of “asthma-related pathologies” include non-asthmatic conditions characterized by airway hyperresponsiveness (eg., chronic bronchitis, emphysema, cystic fibrosis and respiratory distress).

Compositions and methods taught herein are exemplified, for asthma. However, the invention should not be construed as limited to this particular pulmonary disease. Asthma offers the advantage of having been studied extensively and provides several accepted models to evaluate the invention. It is known that sensitization and allergen challenge leads to airway hyperresponsiveness to various agonists. Hence, acetylcholine, known as a spasmogenic agent, capable of inducing larger contractions of the muscle cells in tissues obtained from the trachea of sacrificed animals (which had been sensitized to provoke airway hyper-responsiveness) than from control animals following allergen challenge (see, eg. Tokuoka et at, Br. J. Pharmacol. 0.134:1580 (2001); Nakata et al, Int. Immunol. 13:329 (2001); Emala and Hirshman, Monogr. Allergy 33:35 (1996)).

The most prominent characteristic of asthma is bronchospasm, or narrowing of the airways. Asthmatic patients have prominent contraction of the smooth muscles of large and small airways, increased mucus production, and increased inflammation (Plaut and Zimmerman, sura). The inflammatory response in asthma is typical for tissues covered by a mucosa and is characterized by vasodilation, plasma exudation, recruitment of inflammatory cells such as neutrophils, monocytes, macrophages, lymphocytes, and eosinophils to the sites of inflammation, and the release of inflammatory mediators by resident tissue cells (eg, mast cells or airways epithelial cells) or by migrating inflammatory cells (Hogg, “Pathology of Asthma”, in Asthma as an Inflammatory Disease, O'Byrne (ed.), Marcel Dekker, Inc., New York, N.Y., at 1 (1990)). Asthma may be triggered by a variety of causes such as allergic reactions, a secondary response to infections, industrial or occupational exposures, ingestion of certain chemicals or drugs, exercise (Hargreave et al, J. Allergy Clin. Immunol. 83:1013 (1986)).

AMPA receptor modulators according to the invention have also been found effective to decrease mucus production of bronchial epithelial cells and to inhibit growth factor mediated proliferation of smooth muscle cells.

An increase in bronchial hyperreactivity (AHR), the hallmark of a more severe form of asthma, can be induced by both airway antigenic and non-antigenic stimuli. Late phase response and persistent hyperresponsiveness in allergen-induced asthma have been associated with the recruitment of leukocytes, and particularly eosinophils, to inflamed lung tissue (Abraham et al, Am. Reu Respir. Dis. 138:1565 (1988)). Eosinophils release several inflammatory mediators including 15-HETE, leukotriene C4, PAF, cationic proteins, eosinophil peroxidase.

The endothelins represent an additional group of spasmogenic agents, amongst the most potent vasoconstrictor hormones known. One representative endothelin is endothelin-1 (ET-1), a 21-amino acid peptide produced in a variety of tissues including endothelial and vascular smooth-muscle cells, bronchial epithelial cells, neurons and astrocytes in the central nervous system, as well as endometrial cells. Bronchial epithelial cells respond to IgE-mediated activation with the release of endothelin which suggest a direct role of epithelia in allergic response. Endothelin-1 has a wide range of activities including smooth muscle contraction, mitogenesis, microvascular leakage and edema, mucous gland hypersecretion and neuromodulation leading to its possible involvement in the pathophysiology in different lung diseases (see eg Sokolovsky, Pharmacol. 68:435 (1995); Rubanyi and Polokoff, Pharmacol. Reu 46:325 (1994); Goldie and Fernandes, Monaldi Arch. Chest Dis., 55:162 (2000)). The lung has been shown to be a major source of ET-1 and lung tissues have very high receptor density for this peptide (eg, Hemsen et al., Eur. J. Pharmacol. 191: 319, 1990). Endothelins cause the contraction of lung smooth muscle, an action similar to that experienced during an asthma attack. Stable asthmatics are more sensitive to the bronchoconstrictor effect of ET-1 than non-asthmatics (Chalmers et al, Am. J. Respir. Crit. Care Med. 156:382 (1997)), and it also has been shown that the ET-1 content in arterial blood and bronchoalveolar lavage fluid is higher during an asthma attack and remission compared to healthy controls (Trakada et al., Respir. Med. 94:992 (2000)). Therefore, inhibition of the production or the action of ET-1 has been postulated to relieve asthmatic symptoms. Administration of corticosteroids has been associated with significant fall in ET-1 levels in bronchial biopsy samples and bronchoalveolar lavage fluid as well as in the release of ET-1 from bronchial epithelial cells obtained from asthmatics (Mattoli et al, J. Allergy Clin. Immunol. 88:376 (1991); Redington et al., J. Allergy Clin. Immunol. 100:544 (1997); Vittori et al., Am. Reu Resp. Dis. 146:1320 (1992). It has been discovered that the AMPA receptor modulators according to the invention also reduce ET-1 induced contractions of the lung smooth muscle tissues of animals.

The terms “antigen” and “allergen” are used interchangeably to describe those molecules, such as dust or pollen that can induce an allergic reaction and/or induce asthmatic symptoms in an individual suffering from asthma. Thus, an asthmatic individual “challenged” with an allergen or an antigen is exposed to a sufficient amount of the allergen or antigen to induce an asthmatic response. AMPA receptor modulators according to the invention have been found effective to treat AHR subsequent to ovalburnin sensitization and antigen challenge.

The term “AMPA receptor modulator” is used to designate any compound able to alter the physiological ligand-induced activation state of the receptor. These entities include AMPA receptor agonists, partial agonists, competitive and noncompetitive antagonists of the ligand binding or modulatory site.

The term “AMPA receptor antagonist” is used to designate any moiety down-regulating the activity of the AMPA receptor. Such AMPA receptor antagonist may be either competitive or non-competitive. Non-limiting representative examples of competitive and non-competitive AMPA receptor antagonists are presented below in Tables 1A and 1B, respectively.

Quinoxaline-2,3-diones, including multi-substituted and tricyclic analogs, represent the most important class of compounds in the competitive AMPA receptor antagonist category. The general formula of these compounds can be summarized as

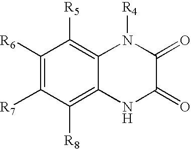

A number of compounds contain an additional fused ring to this structure (eg, bridging R₅ and R₆). Other compounds are in the isatin oxime, quinolon, indenone, amino acid analog and decahydroisoquinoline classes (see Mikai, S., et al., Cur. Med. Chem., 8:155-157 (2001) and Y. B. Auberson, Drugs of the Future 2001, 26(5):463-71 (2001). These are shown in Table 1A.

TABLE 1A
xRepresentative Competitive AMPA Antagonist Receptor Compounds

| | Name | |
|---|---|---|
| Structure | Class | Reference |
| 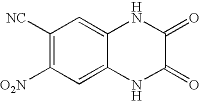 | CNQX Quinoxaline-2,3-dione | Honor\acute{e,}, T. et al., Science 241:701 (1988) |
| 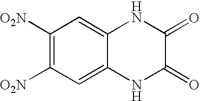 | DNQX Quinoxaline-2,3-dione | Honor\acute{e,}, T. et al., Science 241:701 (1988) |
| 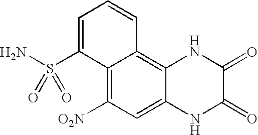 | NBQX Quinoxaline-2,3-dione (tricyclic) | Sheardown et al., Science 247:571 (1990) |
| 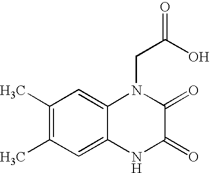 | 1,4-Dihydro-4-carboxymethyl-6,7- dimethyl-quinoxaline- 2,3-dione | Epperrson et al., Bioorg. Med. Chem. Lett. 3:2801 (1993) |
| 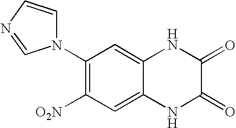 | YM-90K(YM-900) Quinoxaline 2,3-dione | Ohmori et al., J. Med. Chem 37:467 (1994) U.S. Pat. No. 6,096,743 |
| 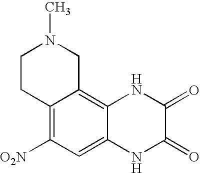 | PNQX (PD 152 247) Quinoxaline-2,3-dione (tricyclic) | Bigge et al., J. Med. Chem. 38:3720 (1995) |
| 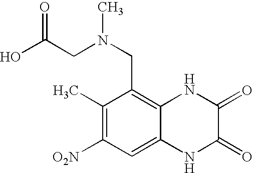 | 5-[N-carboxymethyl,N- methyl)amino-methyl]-6-methyl-7- nitro-quinoxaline-2,3-dione | Auberson et al., Bioorg. Med. Chem. Lett 8:71 (1998) Nikam et al., Curr. Med. Chem. 8:155 (2001) Nikam et al., J. Med. Chem. 42:2266(1999) |
| 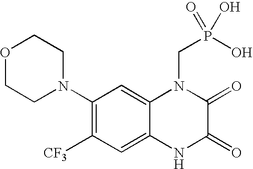 | ZK200775 (Fanampanel) Quinoxaline-2,3-dione | Turski el al., Proc. Natl. Acad. Sci. U.S.A. 95:10960 (1998) |
| 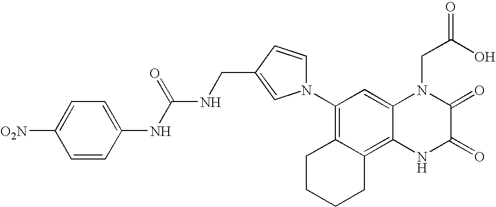 | LU 115455 Quinoxaline-2,3-clione (tricyclic) | Löscher et al., Eur. J. Neurosci. 11:250 (1999) Prog. Neurobiol 54:721 (1998) |
| 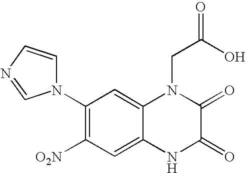 | YM 872 Quinoxaline-2,3-dione | Kohara, A. et al., J. Pharm. Pharmacol 50:795 (1998) |
| 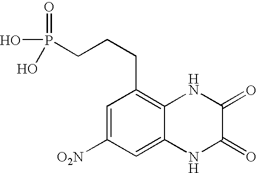 | AMP 397 A Quinoxaline-2,3-dione | Auberson Y.P. et al., 219^{th} ACS Natl Meet (March 26-30, San Franciso) 2000, Abst MEDI-014 |
| 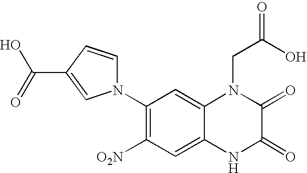 | LU 112313 6-Pyrrolylquinoxaline-2,3-dione | Lubisch W. et al., Bioorg. Med. Chem. Lett. 6:2887 (1996) Lubisch W. et al., Bioorg. Med. Chem. Lett. 7:1101 (1997) |
| 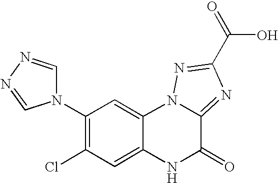 | TQX-173 Triazolo-quinoxaline-carboxylate | Catarzi et al., J. Med. Chem. 43:3824 (2000) |
| 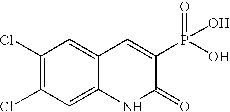 | S-17625 2(1H)-oxoquinoline analog | Desos et al., J. Med Chem. 39:197 (1996) |
| 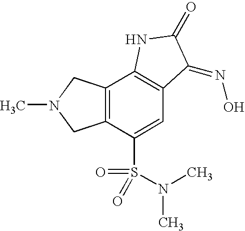 | NS 257 Isatine oxime | Wätjen, F. et al., Bioorg. Med. Chem. Lett. 4:371 (1994) |
| 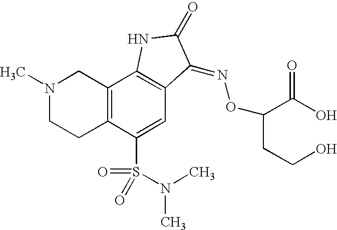 | NS-1209 (SPD 502) Isatine oxime | Nielsen, E.O. et al., J. Pharmacol. Exp. Ther. 289:1492 (1999) WO98/ 14447 Nikam et al. Curr. Med. Chem. 8:155 (2001) |
| 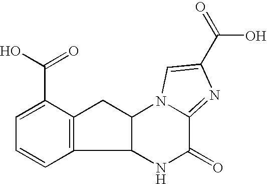 | Indenone 4 f Imidazo-indeno-pyrazine | Mignani et al., Bioorg. Med. Chem. Lett. 10:2749-2759 (2000) U.S. Pat. No. 5,672,705 |
| 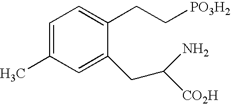 | 2.Phosphonoethyl-5-methyl- phenylalanine Amino acid analog | Hamilton, G.S. et al., Bioorg Med. Chem. Lett. 2:1269 (1962) |
| 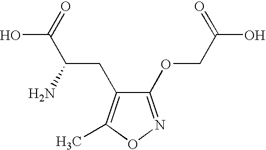 | (S)-AMOA Amino acid analog | Bang-Andersen et al., J. Med. Chem. 40:2831 (1997) Madsen, U. et al, Eur. J. Med. Chem 35:69 (2000) US. Pat. No. 6,200,999 |
| 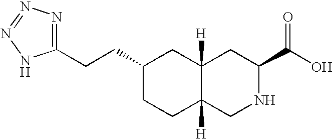 | LY-293558, LY-326325 Decahydroisoquinoline analog | Ornstein et al., J. Med Chem. 36:2046 (1993) |

2,3-benzodiazepines constitute a representative class of noncompetitive AMPA antagonists. They are based on the following structure:

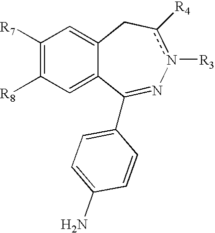

Related compounds include phthalazine derivatives, which can be considered as structurally similar with a six-membered ring replacing the seven-membered ring of the benzodiazepines. Structurally less related aryl-quinazoline-4-one and oxadiazolyl-phenoxy-ethylamine compounds also have been discovered. Nonlimiting representative compounds are shown below in Table 1B.

TABLE 1B
Representative Noncompetitive AMPA Receptor Antagonists

| | Name | |
|---|---|---|
| Structure | Class | Reference |
| 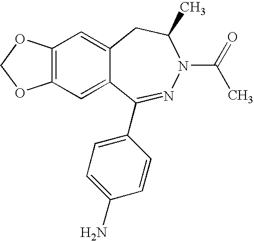 | GYKI-52466 2,3-Benzodiazepine | Tarnawa. et al., Eur. J. Pharmacol 167:193 (1989) |
| 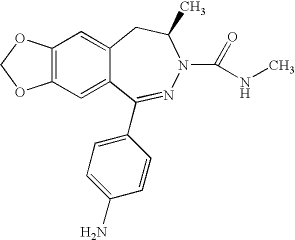 | racern: GYKI-53405 (−) enant.: GYKI-53773 (LY-300164) Talampanel Dihydro-2,3-benzodiazepine | Tarnawa. et al., Bioorg Med. Chem. Lett. 3:99 (1993) U.S. Pat. No. 5,536,832 |
| 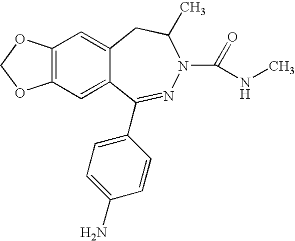 | racem: GYKI-53655 (−) enant.: GYKI-53784 Dihydro-2,3-benzodiazepine | U.S. Pat. No. 5,536,832 |
| 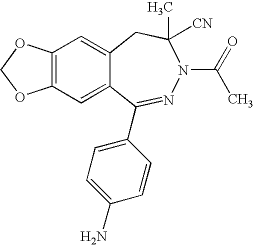 | Egis 8332 Dihydro-2,3-benzodiazepine | U.S. Pat. No. 5,807,851 U.S. Pat. No. 6,075,018 |
| 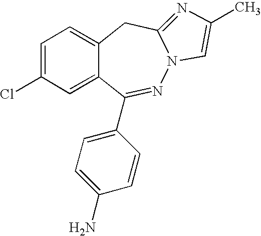 | GYKI-47261 Annulated 2,3-benzodiazepine | Ábramhám et al., Bioorg Med. Chem. 8:2127 (2000) WO99/06408 |
| 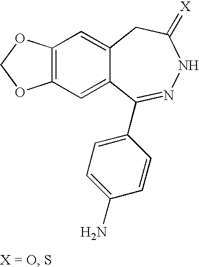 | 1-(4'-Aminophenyl)-3,5-dihydro-7,8- methylenedioxy-4H-2,3- benzodiazepine- 4-one or 4-thione | Wang et al., J. Med. Chem. 41:2621 (1998) De Sarro et al., Bioorg, Med. Chem. Lett. 8:971 (1998) Grasso et al., J. Med. Chem. 42:4414 (1999) |
| 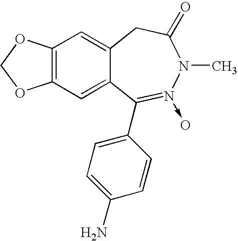 | 3-Methyl-7,8-methylenedioxy-1-(4- aminophenyl)-3,5-dihydro-2,3- benzodiazepin-4(4H)-one 2,3-Benzodiazepine-4-one | U.S. Pat. No. 5,891,871 |
| 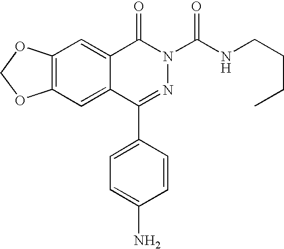 | SYM 2207,SYM 2189 1,2-Dihyroplithalazine | Pelletier et al., J. Med. Chem. 39:343 (1996) Pei et al., Bioorg Med. Chem. Lett. 9:539 (1999) U.S. Pat. No. 5,716,956 |
| 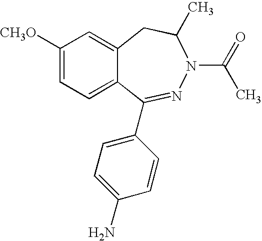 | SYM-2267 3,5-Dihydro-5H-2,3- benzodiazepine | WO01/98280 A2 |
| 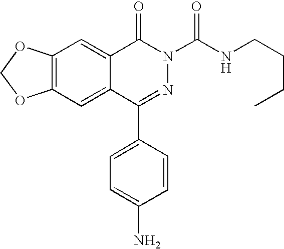 | 4-(Aminophenyl)-6,7- (methylenedioxy)-N-butyl-1,2- dihydrophthalazine-1-one-2- carboxamide Phthalazinone | Grasso et al., J. Med Chem. 43:2851 (2000) |
| 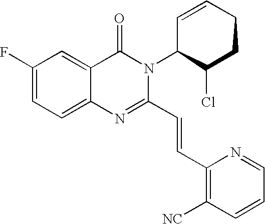 | CP-526,427 Quinazoline-4-one derivatives | Chenard et al., Bioorg. Med. Chem. Lett. 10:1203 (2000) Menniti et al., Mol. Pharmacol. 58:1310 (2000) Chenard et al., J. Med. Chem. 44:1710 (2001) U.S. Pat. No. 6,060,479 |
| 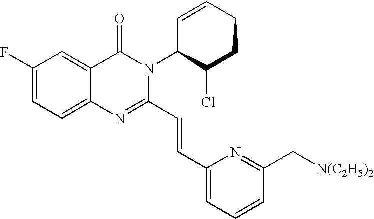 | CP-465,022 Quinazoline-4-one derivatives | Welch et al., Bioorg. Med. Chem. Lett. 11:177 (2001) Lazarro, J.T. et al., Neuropharmacology 42:143 (2002) |
| 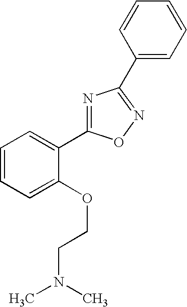 | BIIR 561 CL (Irampanel) Oxadiazolyl-phenoxy-ethylamine (also voltage-deplendent sodium channel antagonist) | Weiser et al., J. Pharmacol. Exp. Ther. 289:1343 (1999) |

Several non-competitive AMPA receptor antagonists are known in the literature in conjunction with non-lung tissues (see eg, Nikam et al, Current Medicinal Chemistry 8:155 (2001) and the references listed above in Table 1B). They encompass a variety of compounds that have been found to modulate the activity of the AMPA receptor by interacting with at least one allosteric binding site of the receptor.

Administration may be as a single or divided dose.

Examples of non-competitive AMPA receptor antagonist include, without limitation, 1-(4-aminophenyl)-4-methyl-7,8-methylenedioxy-5H-2,3-benzodiazepine (GYKI 52466); 1-(4-aminophenyl)-3-acetyl-4-methyl-3,4-dihydro-7,8-methylenedioxy-5H-2,3-benzodiazepine (GYKI-53405); 1-(4-aminophenyl)-8-chloro-2-methyl-1 1H-imidazo[1,2-c][2,3] benzodiazepine (GYKI-47261); (−)-1-(4-aminophenyl)-3-acetyl-4-methyl-7,8-methylenedioxy-3,4-dihydro-5H-2,3-benzodiazepine (GYKI 53773, talampanel); (−)-1-(4-aminophenyl)-3-methylcarbamoyl-4-methyl-7,8-methylenedioxy-3,4-dihydro-5H-2,3-benzodiazepine (GYKI-53784); 3-(2-chlorophenyl)-2-[2-(3-cyano-pyridin-2-yl)-vinyl]-6-fluoro-3-quinazoline-4-one (CP-526,427); (S)-3-(2-chlorophenyl)-2-[2-(6-diethylaminomethyl-pyridin-2-yl)-vinyl]-6-fluoro-3H-quinazoline-4-one (CP-465,022); 1-(4-amino-phenyl)-3,5-dihydro-4-methyl-3-acetyl-7-methoxy-5H-2,3-benzodiazepine (SYM 2267); N,N-dimethyl-2-[2-(3-phenyl-1,2,4-oxadiazol-5-yl)phenoxy]ethanamine (BIIR 561 CL, irampanel) and pharmaceutically acceptable salts thereof. Non-competitive AMPA receptor antagonists contemplated include GYKI-52466; GYKI-53405; GYKI-53655; GYKI-53773; GYKI-53784; GYKI-47261; CP-526,427; CP-465,022; SYM-2267 and BIIR 561 CL. Alternatively, in other embodiments non-competitive AMPA receptor antagonists useful according to the invention are: GYKI-52466, GYKI-47261, GYKI-53773 and GYKI-53784.

The AMPA receptor antagonist may alternatively be a competitive AMPA receptor antagonist, such as the compounds listed hereinbefore in Table 1A. Competitive AMPA receptor antagonists include CNQX; DNQX; NBQX; PNQX (PD 152247); NS-1209 (SPD-502); 5-[N-carboxymethyl, N-methyl)aminomethyl]-6-methyl-7-nitro-quinoxaline-2,3-dione; LU 115 455; YM 872 (Zonampanel); AMP 397A; LU 112 313; TQX-173; indenone 4f; 2-phosphonoethyl-5-methyl-phenylalanine; [6,7-dichloro-2(1″)oxoquinoline-3-yl]phosphoric acid (S-17625), (S)-AMOA and LY-293558 (LY-326325). In yet other embodiments contemplated competitive AMPA antagonists useful according to the invention are: NS-1209 (SPD-502); 5-[N-carboxymethyl,N-methyl)aminomethyl]-6-methyl-7-nitro-quinoxaline-2,3-dione; YM 872 (Zonampanel); AMP 397A; and LU 112 313.

An AMPA receptor antagonist to be used in the present invention, which is basic in nature, is capable of forming a wide variety of different salts with various inorganic and organic acids. Although such salts must be pharmaceutically acceptable for administration to animals, it is often desirable in practice to initially isolate said AMPA receptor antagonist from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of the method of this invention are prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare a pharmaceutically acceptable acid addition salt of an AMPA receptor antagonist to be used in the present invention are those which form non-toxic acid addition salts, ie, salts containing pharmacologically acceptable anions, (see Remington references Ha) such as for example hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, sacharate, benzoate, methanesulfonate and pamoate [i.e, 1,1′-methylene-bis-(2-hydroxy-3-naphthoate)] salts.

Those AMPA receptor antagonists to be used in the present invention, which are acidic in nature, are capable of forming base salts with various pharmacologically acceptable cations. Examples of such salts include the alkali metal or alkaline-earth metal salts and in particular, the sodium and potassium salts. These salts are all prepared by conventional techniques. The chemical bases which are used as reagents to prepare the pharmaceutically acceptable base salts of this invention are those, which form non-toxic, base salts with the AMPA receptor antagonists to be used in the present invention. These non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction of maximum product of yields of the desired final product.

The methods of the present invention are intended for use with any mammal that may experience the benefits of the methods of the invention. Foremost among such mammals are humans, although the invention is not intended to be so limited, and is applicable to veterinary uses. Thus, in accordance with the invention, “mammal” or “mammal in need” include humans as well as non-human mammals, particularly domesticated animals including, without limitation, cats, dogs, and horses.

The term “therapeutically effective amount” is used to denote treatments at dosages effective to achieve the therapeutic result sought. Furthermore, one of skill will appreciate that the therapeutically effective amount of the compound of the invention may be lowered or increased by fine-tuning and/or by administering more than one compound of the invention, or by administering a compound of the invention with another anti-asthmatic compound (eg, corticosteroid). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect. Clinical changes relevant to assess the therapeutic effect of treatment according to the invention include reduction in the characteristic symptoms and signs of asthma and related pathologies (eg, dyspnea, wheezing, cough, bronchial hypersensitivity airway remodeling) and improvement of pulmonary function tests. These are based upon patient's symptoms and physician's observations.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

Contemplated therapeutically effective amounts therefore, are from about 0.01 mg/kg/day to about 300 mg/kg/day when administered systemically (eg., orally administered). In an embodiment of the invention, when systemically administered, therapeutically effective amounts are from about 0.1 mg/kg/day to about 150 mg/kg/day.

For local administration by inhalation for example, contemplated therapeutically effective amounts are from about 0.01 μg/kg/day to about 300 μg/kg/day when administered systemically (eg., orally administered). In an embodiment of the invention, when systemically administered, therapeutically effective amounts are from about 0.1 μg/kg/day to about 150 μg/kg/day.

Dosage forms and frequency of administration of the same, will depend on conventional factors routinely considered by one of skill in the field to obtain therapeutically effective amounts as discussed above in a given mammal. Hence, a practitioner will consider the condition being treated, the particular compound of the invention being administered, route of administration, and other clinical factors such as age, weight and condition of the mammal as well as convenience and patient compliance.

It will be appreciated by those of skill in the art that the number of administrations of the compounds according to the invention will vary from patient to patient based on the particular medical status of that patient at any given time.

When applicable (such as for the treatment of asthma, for example) the compound according to this aspect of the invention, may be administered prior to, at the same time, or after the mammal has been exposed to an antigen. In addition, the timing of the administration of the compound of the invention with relation to the exposure to an antigen will vary from mammal to mammal depending on the particular situation. A skilled practitioner will optimize administration by careful monitoring the patient while altering the timing and/or the order of administration of the compound of the invention. Hence, it will be understood that the mammal need not suffer from a pulmonary inflammation to benefit from the invention. The compounds of the invention may be administered prophylactically to individuals predisposed to develop asthma and/or an asthma-related pathology. For example, an individual allergic to pollen may be administered a compound of the invention (eg., by oral administration) on a daily basis and/or prior to going to a pollen-rich area (eg, a garden). Likewise, an individual with only a family history of asthmatic attacks may be administered the compounds of the invention prophylactically—to prevent possible onset of such an asthmatic attack.

The compounds according to the invention are optimally formulated in a pharmaceutically acceptable vehicle with any of the well-known pharmaceutically acceptable carriers, including diluents and excipients (see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention, as well as any other pharmacologically active ingredient useful for the treatment of the particular pulmonary inflammation being treated. Such compounds may include without limitation, β-andrenoceptor antagonists: albuterol, metaproteranol, levealbuterol, pirbuterol, salmeterol, bitolterol; glucocorticoids: beclamethasone, triamcinolone, flunisolide, budesonide, fluticasone; leukotriene-receptor antagonists and leukotriene-synthesis inhibitors: zafirlukast, montelukast, zileutin; other anti-asthmatics: cromolyn, nedocroril, theophylline; anti-cholinergic agents: ipatropium, oxitropium, tiotropium; H₁ receptor antagonist anti-histamines: diphenydramine, pyrilamine, promethazine, loratidine, chlorocyclizine, chlorophemiramine, fexofenadine; adrenocorticosteroids; and glucocorticoids.

The compositions of the invention can be administered by standard routes (eg. oral, inhalation, rectal, nasal, topical, including buccal and sublingual, or parenteral, including subcutaneous, intramuscular, intravenous, intradermal, transdermal, and intratracheal). In addition, polymers may be added according to standard methodologies in the art for sustained release of a given compound.

Formulations suitable for administration by inhalation include formulations that can be dispensed by inhalation devices known to those in the art. Such formulations may include carriers such as powder and aerosols. The present invention encompasses liquid and powdered compositions suitable for nebulization and intrabronchial use, or aerosol compositions administered via an aerosol unit dispensing metered doses (“MDI”). Particularly preferred devices contemplated are described in U.S. Pat. No. 5,447,150.

The active ingredient may be formulated in an aqueous pharmaceutically acceptable inhalant vehicle, such as, for example, isotonic saline or bacterostatic water and other types of vehicles that are well known in the art. The solutions are administered by means of a pump or squeeze-actuated nebulized spray dispenser, or by any other conventional means for causing or enabling the requisite dosage amount of the liquid composition to be inhaled into the patient's lungs.

Powder compositions containing the anti-inflammatory compounds of the present invention include, by way of illustration, pharmaceutically acceptable powdered preparations of the active ingredient thoroughly intermixed with lactose or other inert powders acceptable for intrabronchial administration. The powder compositions can be administered via a dispenser, including, but not limited to, an aerosol dispenser or encased in a breakable capsule, which may be inserted by the patient into a device that punctures the capsule and blows the powder out in a steady stream.

Aerosol formulations for use in the subject method typically include propellants, surfactants, and co-solvents and may be filled into conventional aerosol containers that are closed by a suitable metering valve.

For oral administration, the anti-inflammatory compositions of the invention may be presented as discrete units such as capsules, caplets, gelcaps, cachets, pills, or tablets each containing a predetermined amount of the active ingredient as a powder or granules; as a solution or a suspension in an aqueous liquid or a non-aqueous liquid; or as an oil-in-water liquid emulsion or a water-in-oil emulsion and as a bolus, etc Alternately, administration of a composition of all of the aspects of the present invention may be effected by liquid solutions, suspensions or elixirs, powders, lozenges, micronized particles and osmotic delivery systems.

Formulations of compositions of the present invention suitable for nasal administration, wherein the carrier is a solid, include a coarse powder having a particle size, for example, in the range of 20 to 500 microns which is administered in the manner in which snuff is administered, ie by rapid inhalation through the nasal passage from a container of the powder held close up to the nose. Suitable formulations, wherein the carrier is a liquid, for administration, for example via a nasal spray, aerosol, or as nasal drops, include aqueous or oily solutions of the compound of the invention.

Formulations of compositions suitable for parenteral administration include aqueous and non-aqueous sterile injection solutions which may contain antioxidants, stabilizers, buffers, bacteriostats and solutes which render the formulation isotonic with the blood of the intended recipient; and aqueous and non-aqueous sterile suspensions which may include suspending agents and thickening agents.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLE 1

### Investigation for the Presence/Function of AMPA Receptor in Rat Lung

The presence/function of AMPA receptors in the lung of experimental animals was investigated in the same model (ventilated perfused rat lung) that was applied for the examination of NMDA-receptors by Said et al., (Proc Natl. Acad. Sci. (USA) 93:4688 (1996). The lung of anesthetized SPRD rats were ventilated and were in situ perfused with a constant flow of Krebs solution. L-arginine, AMPA and AMPA-antagonist were added directly to the perfusate as described by Said et al, supra. As it is shown in Table 2, activation of the AMPA receptors triggered an acute edematous lung injury manifested by the significant increase in airway pressure (PAW) pulmonary artery pressure (PAP) and the ratio of wet and dry weight of the lung after the treatment. Administration of GYKI-53784 decreased both the airway and the pulmonary arterial pressures.

TABLE 2
Effect of AMPA and a non-competitive AMPA receptor
antagonist, GYKI 53784-treatment on different parameters
of pulmonary functions in ventilated, perfused lung of SPRD-rats.

| | Treatment (n) | | |
|---|---|---|---|
| | | | AMPA |
| | | | (200 μM) + 53784 |
| Parameters | Control | AMPA (200 μM) | (200 μM) |
| P_{AW}* | 99.3 ± 4.9 (8) | 183.4 ± 15.6 (7) | 110.3 ± 19.1 (4) |
| p | | <0.001^{a} | 0.020^{b} |
| P_{AP}** | 96.0 ± 8.2 (9) | 147.9 ± 8.6 (7) | 114.0 ± 23.0 (4) |
| p | | 0.001^{a} | NS^{b} |
| W/D-ratio*** | 6.6 ± 0.4 (3) | 14.8 ± 3.9 (3) | 14.3 ± 5.6 (4) |
| p | | NS^{a} | NS^{b} |

*P_{AW}: Airway pressure after 60 minute perfusion as a percent of 0 minute pressure
**P_{AP}: Pulmonary artery pressure after 60 minutes perfusion as a percent of 0 minute pressure
***W/D: Ratio of wet and dry weight of lungs after 60-minute perfusion
^{a}compared to control
^{b}compared to AMPA-treatment

### EXAMPLE 2

### Investigation of the Effect of Orally Administered Non-Competitive AMPA Receptor Antagonist on Airways' Hyperresponsiveness ex Vivo

Inflammation of the airways may lead to bronchial hyper-responsiveness, which is a characteristic feature of asthma.

Rats were actively sensitized to ovalbumin (OA) by a subcutaneous injection of 0.5 ml of OA/Al(OH₃) gel mixture (2 mg OA+10 g Al(OH₃)/100 ml saline) on day 1 with subsequent subcutaneous injections (10 mg OA+10 g Al(OH₃)/100 ml saline) given on days 14 and 21. On day 28, animals received GYKI-52466, GYKI-47261 or GYKI-53773 compound orally (0.3 mg/kg or 3.0 mg/kg) 2 hours before antigen challenge. Antigen challenge was performed by inhalation of nebulised ovalbumin (1% antigen solution administered in a TSE inhalation system for 1 hour). Animals were sacrificed 48 hours post antigen challenge wherein the tracheas were removed to an organ bath. Dissected tracheas were allowed to equilibrate for 30 minutes before measuring tracheal spasmogenic response curves to acetylcholine (Ach).

As shown in Table 3 ovalbumin challenge of sensitized animals in this model caused a significant tracheal hyper-reactivity to acetyicholine, when the response to the spasmogenic agent was determined 48 h after antigen challenge. GYKI 52466, GYKI-47261 and GYKI 53773 in a dose of 3.0 mg/kg, brought this elevation back to control level.

TABLE 3
Effect of antigen challenge and oral pretreatment with different GYKI
compounds on the tracheal contraction to acetylcholine in BN-rats

| | | | | GYKI-47261 | GYKI-53773 |
|---|---|---|---|---|---|
| Parameters | Control | Challenged | GYKI-52466 3.0 mg/kg | 3.0 mg/kg | 3.0 mg/kg |
| ED₅₀* | 5.63 ± 0.11 | 6.74 ± 0.32 | 5.60 ± 0.46 | | |
| p | 0.002 | | 0.028 | | |
| MAX** | 100 ± 0 | 276 ± 47 | 135 ± 32 | | |
| p | 0.001 | | 0.037 | | |
| ED₅₀* | 5.22 ± 0.14 | 5.89 ± 0.18 | | 5.26 ± 0.30 | 4.64 ± 0.34 |
| p | 0.003 | | | 0.047 | 0.001 |
| MAX** | 100 ± 0 | 163 ± 18 | | 98 ± 13 | 85 ± 16 |
| p | <0.001 | | | 0.020 | 0.007 |

*−log M acetylcholine (Ach) causing 50% contraction relative to control (mean ± SEM)
**Contraction at maximal Ach concentration relative to control (mean ± SEM)

### EXAMPLE 3

### Investigation of the Effect of Locally Administered Non-Competitive AMPA Receptor Antagonist on Airways' Hyperresponsiveness ex Vivo

The procedures of Example 2 were followed in order to compare the effects of antigen exposure and local (intratracheal) GYKI 53773 treatments on Ach-induced tracheal contractions in allergen sensitized and challenged rats. The results, obtained with GYKI-53773 (Talampanel), shown in Table 4 clearly demonstrate that the examined compound when administered intratracheally strongly reduced the allergen-induced airways hyperresponsiveness at a very low (1 μg/kg) dose even after a single administration.

TABLE 4
Effect of antigen challenge and local (intratracheal) treatment
with the non-competitive AMPA receptor antagonists (Talampanel)
on the tracheal contraction to acetylcholine in BN-rats

| | | | | GYKI-53773 | GYKI-53773 |
|---|---|---|---|---|---|
| Parameters | Control | Challenged | GYKI-53773 mg/kg | mg/kg | mg/kg |
| ED₅₀* | 5.89 ± 0.12 | 7.22 ± 0.24 | 5.80 ± 0.47 | 5.73 ± 0.39 | 5.68 ± 0.55 |
| p | <0.001 | | 0.011 | 0.004 | 0.013 |
| MAX** | 100 ± 0 | 312 ± 99 | 202 ± 66 | 113 ± 19 | 129 ± 30 |
| p | 0.011 | | NS‡ | 0.036 | 0.053 |

*−log M acetylcholine (Ach) causing 50% contraction relative to control (mean ± SEM)
**Contraction at maximal Ach concentration relative to control (mean ± SEM)
‡Non-significant (p > 0.05)

### EXAMPLE 4

### Investigation of the Effect of a Competitive AMPA Receptor Antagonist, Administered Systemically (Intraperitoneally), on Airways' Hyperresponsiveness ex Vivo

The procedures of Example 2 were followed in order to compare the effects of antigen exposure and systemic (intraperitoneal) NBQX treatments on Ach-induced tracheal contractions in allergen sensitized and challenged rats.

The data of Table 5 demonstrate that the competitive AMPA-antagonist NBQX strongly inhibits both parameters (ED₅₀ and maximal provoked response) of allergen-induced airways hypersensitivity of the experimental animals in a dose-dependent way.

TABLE 5
Effect of antigen challenge and systemic (intraperitoneal) treatment with the competitive
AMPA receptor antagonist NBQX on the tracheal contraction to acetylcholine in BN-rats

| | | | | NBQX | NBQX |
|---|---|---|---|---|---|
| Parameters | Control | Challenged | NBQX 0.15 mg/kg | 1.5 mg/kg | 15 mg/kg |
| ED₅₀* | 6.16 ± 0.31 | 7.31 ± 0.37 | 6.29 ± 0.51 | 4.98 ± 0.80 | 5.11 ± 0.55 |
| p | 0.015 | | NS‡ | <0.011 | <0.001 |
| MAX** | 100 ± 0 | 308 ± 34 | 178 ± 65 | 115 ± 30 | 125 ± 32 |
| p | <0.001 | | NS‡ | <0.001 | <0.001 |

*−log M acetylcholine (Ach) causing 50% contraction relative to control (mean ± SEM)
**Contraction at maximal Ach concentration relative to control (mean ± SEM)
‡Non-significant (p > 0.05)

### EXAMPLE 5

### Investigation of the Effect of a Competitive AMPA Receptor Antagonist, Administered Locally (Intratracheally) on Airways' Hyperresponsiveness ex Vivo

The procedures of Example 2 were followed in order to compare the effects of antigen exposure and systemic (intraperitoneal) NBQX treatments on Ach-induced tracheal contractions in allergen sensitized and challenged rats. The data of Table 6 demonstrate that the competitive AMPA-antagonist NBQX inhibits allergen-induced airways hypersensitivity in a dose-dependent manner, however, it must be noted that its effect is statistically significant only at the highest applied dose.

TABLE 6
Effect of antigen challenge and local treatment with the competitive AMPA receptor
antagonists (NBQX) on the tracheal contraction to acetylcholine in BN-rats

| | | | | NBQX | NBQX |
|---|---|---|---|---|---|
| Parameters | Control | Challenged | NBQX Mg/kg | mg/kg | mg/kg |
| ED₅₀* | 5.68 ± 0.12 | 6.70 ± 0.55 | 5.85 ± 0.59 | 5.20 ± 0.72 | 3.44 ± 0.36 |
| p | 0.010 | | NS‡ | NS‡ | <0.001 |
| MAX** | 100 ± 0 | 327 ± 133 | 308 ± 128 | 108 ± 28 | 60 ± 14 |
| p | 0.006 | | NS‡ | NS‡ | 0.025 |

*−log M acetylcholine (Ach) causing 50% contraction relative to control (mean ± SEM)
**Contraction at maximal Ach concentration relative to control (mean ± SEM)
‡Non-significant (p > 0.05)

### EXAMPLE 6

### Investigation of the Effect of AMPA-Modulators on ET-1 Provoked Tracheal Contraction in Vitro

In order to measure the effect of GYKI 52466 and 53773 (talampanel) on endothelin-1-induced contractions in isolated guinea pig trachea the following procedures were followed. The tracheae of male Dunkin-Hartley guinea pigs (350-450 g) were removed; helical strips were prepared and mounted in an organ bath. After equilibration, the strips were contracted with endothelin-1 and when contraction had reached the plateau, different concentrations of GYKI-52466, 53773 or the suitable vehicles were added into the chambers. Changes in tensions were expressed as the percentage of the maximal response to 4.5 μM histamine measured at the beginning of the experiment.

The results obtained, shown in Table 7, demonstrate that both GYKI-52466 and talampanel can dose-dependently inhibit the contraction caused by ET-1 in the isolated guinea pig trachea.

TABLE 7
Effect of AMPA receptor modulators on the endothelin
induced tracheal contractions in vitro

| | | Contraction %* | |
|---|---|---|---|
| Experiment | Treatment | Mean ± SD (n) | Significance** |
| 1 | Vehicle | 84 ± 8 (5) | <0.05 |
| | 52466 (10 μM) | 53 ± 7 (5) | |
| 2 | Vehicle | 66 ± 8 (5) | <0.01 |
| | 52466 (30 μM) | 23 ± 9 (5) | |
| 3 | Vehicle | 84 ± 8 (5) | <0.05 |
| | 53773 (10 μM) | 43 ± 9 (8) | |
| 4 | Vehicle | 66 ± 8 (5) | <0.01 |
| | 53773 (30 μM) | 7 ± 4 (4) | |

*Contraction percent of maximal response to 4.5 μM histamine is shown after 25 minutes of addition of the AMPA receptor modulator
**ANOVA followed by Dunnett-test, compared to vehicle-treatment

### EXAMPLE 7

### Investigation of the Effect of AMPA Receptor Modulators on Allergen-Induced Mucus Production of Airways Epithelial Cells

Antigen sensitized BN rats were pretreated orally with 0.3, 3 and 30 mg/kg GYKI-52466 or GYKI-53773 (Talampanel) 2 hours prior to antigen challenge using a similar protocol that was described in Example 2. Lungs were isolated 48 hours subsequent to antigen exposure; samples were fixed in phosphate-buffered 8% formalin. Specimens were then routinely processed for histochemistry. In the periodic acid-Schiff stained (PAS) and haematoxylin-eosin counterstained, 5 μm thick sections, all epithelial cells of each airway segments were counted in the whole preparations at a magnification of 400×. The number of PAS(+) [mucus-producing] epithelial cells are expressed as the ratio of all epithelial cells.

As it is shown in Table 8, allergenic challenge stimulates mucus production of airways epithelial cells (control vs. challenge). Both compounds statistically significantly decreased the number of mucus-producing (PAS(+))epithelial cells, even at the smallest applied dose, and showed dose-dependence in their effect which was especially remarkable in case of talampanel.

TABLE 8
Effect of oral treatment with non-competitive AMPA
receptor antagonists on the allergen-induced mucus
production of airways' epithelial cells in BN-rats

| Treatment | | %* | p-value |
|---|---|---|---|
| Control | | 0.55 | — |
| Challenged | | 16.04 ± 1.83 | <0.001 |
| 53773 | 0.3 mg/kg | 12.34 ± 1.39 | 0.03 |
| | 3 mg/kg | 9.49 ± 1.08 | 0.03 |
| | 30 mg/kg | 5.02 ± 0.75 | <0.001 |
| Control | | 2.36 ± 0.49 | — |
| Challenged | | 26.04 ± 2.50 | <0.001 |
| 52466 | 0.3 mg/kg | 16.09 ± 1.82 | 0.006 |
| | 3 mg/kg | 15.18 ± 1.68 | 0.007 |
| | 30 mg/kg | 14.17 ± 1.59 | <0.001 |

*% PAS(+) cells of total epithelial cells (mean ± SEM)

### EXAMPLE 8

### Investigation of the Effect of AMPA Receptor Modulators on Growth Factor-Stimulated Proliferation of Smooth Muscle Cells

Primary and permanently cultured rat aortic smooth muscle cells were made senescent by serum deprivation (0.1% FCS, 48 hours). Proliferation was induced by re-addition of serum (10%) containing the necessary growth factors and the effect of different concentrations of the AMPA receptor modulators on the proliferation of the cells was determined by measuring ³H-thymidine incorporation.

As it is shown in Table 9, the examined AMPA receptor modulators significantly inhibited growth factor induced proliferation of rat smooth muscle cells, under micro molar concentrations.

TABLE 9
Effect of AMPA receptor modulators on the growth
factor-induced proliferation of smooth muscle cells in vitro

| | |
|---|---|
| | Inhibition |
| Concentration | (% of control) |

| Compound | (μg/ml) | RASMC* | A7r5** |
|---|---|---|---|
| 53773 | 0.03 | 26 | 21 |
| | 0.3 | 59 | 43 |
| | 3.0 | 70 | 87 |
| NBQX | 0.03 | 1 | NE*** |
| | 0.3 | 5 | NE |
| | 3.0 | 94 | NE |

*Rat aortic smooth muscle cells (primary)
**Established smooth muscle cell line (ATCC)
***Not examined

### Equivalents

While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A method for treating an inflammatory disorder of the airways in a mammal, comprising the step of administering to a mammal a therapeutically effective amount of an AMPA receptor modulator compound.

2. The method of claim 1, wherein the AMPA receptor modulator is a non-competitive AMPA receptor antagonist.

3. The method of claim 2, wherein the non-competitive AMPA receptor antagonist is selected from the group consisting of 1-(4-aminophenyl)-4-methyl-7,8-methylenedioxy-5H-2,3-benzodiazepine (GYKI 52466); 1-(4-aminophenyl)-3-acetyl-4-methyl-3,4-dihydro-7,8-methylenedioxy-5H-2,3-benzodiazepine (GYKI-53405); 1-(4-aminophenyl)-8-chloro-2-methyl-1H-imidazo[1,2-c][2,3] benzodiazepine (GYKI-47261); (−)-(4-aminophenyl)-3-acetyl-4-methyl-7,8-methylenedioxy-3,4-dihydro-5H-2,3-benzodiazepine (GYKI 53773, talampanel); (−)-1-(4-aminophenyl)-3-methylcarbamoyl-4-methyl-7,8-methylenedioxy-3,4-dihydro-5H-2,3-benzodiazepine (GYKI-53784); 3-(2-chlorophenyl)-2-[2-(3-cyano-pyridin-2-yl)-vinyl]-6-fluoro-3-quinazoline-4-one (CP-526,427); (S)-3-(2-chlorophenyl)-2-[2-(6-diethylaminomethyl-pyridin-2-yl)-vinyl]-6-fluoro-3H-quinazoline-4-one (CP-465,022); 1-(4-amino-phenyl)-3,5-dihydro-4-methyl-3-acetyl-7-methoxy-5H-2,3-benzodiazepine (SYM 2267); N,N-dimethyl-2-[2-(3-phenyl-1,2,4-oxadiazol-5-yl)phenoxy]ethanamine (BIIR 561 CL, irampanel) and pharmaceutically acceptable salts thereof.

4. The method of claim 1, wherein the AMPA receptor modulator is a competitive AMPA receptor antagonist.

5. The method of claim 4, wherein the competitive AMPA receptor antagonist is selected from the group consisting of 6-cyano-7-nitroquinoxaline-2,3-dione (CNQX); 6,7-dinitroquinoxaline-2,3-dione (DNQX); 2,3-dihydroxy-6-nitro-7-sulfamoyl-benzo(f)quinoxaline (NBQX); 1,4,7,8,9,10-hexahydro-9-methyl-6-nitropyrido[3,4-f]quinoxaline-2,3-dione (PNQX, PD 152247); 8-methyl-5-[4-(dimethylsulfamoyi)phenyl]-6,7,8,9-tetrahydro-1H-pyrrolo[3,2-h]isoquinoline-2,3-dione-3-G (4-hydroxybutiric acid)oxine (NS-1209,SPD-502); 5-[(N-carboxymnethyl,N-methyl)amino-methyl]-6-methyl-7-nitro-quinoxaline-2,3-dione; N-((1-(1-carboxymethyl-5,6,7,8-tetrahydro-benzo(f)quinoxaline-2,3-(1H,4H)-dion-9-yl)pyrrol-3-yl)methyl-N′-(4-carboxyphenyl)-urea (LU 115 455); 1,4-dihydro-4-carboxymethyl-6-(1H-imidazol-1-yl)-7-nitro-2,3-quinoxaline-dione (YM 872, zonampanel); 5-[N-(phosphono-methyl)amino-methyl]-7-nitroquinoxaline-2,3-dione (AMP 397A); 1-carboxymethyl-7-(3-carboxypyrrol-1-yl)-6-nitroquinoxaline-2,3-(1H,4H)-dione (LU 112 313); 7-chloro-4,5-dihydro-8-(1,2,4-triazol-4-yl)-4-oxo-1,2,4-triazolo[1,5-a]quinoxaline-2-carboxylic acid (TQX-173); [6,7-dichloro-2(1H)oxoquinoline-3-yl]phosphonic acid (S-17625); 9-carboxymethyl-4,5-dihydro-4-oxo-imidazo[1,2-a]indeno[1,2-e]pyrazin-2-carboxylic acid (Indenone 4f); 2-phosphonoethyl-5-methyl-phenylalanine; 2-amino-3-[(3-carboxymethoxy)-5-methylisoxazol-4-yl]propionate [(S)-AMOA]; (3S,4aR,6R,8aR)-decahydro-6-[2-(1H-tetrazol-5-yl)ethyl]-3-isoquinoline-3-carboxylic acid (LY-293558, LY-326325) and pharmaceutically acceptable salts thereof.

6. The method of claim 1, wherein the inflammatory disorder of the airways is an allergic inflammatory disorder of the airways.

7. The method of claim 6, wherein the allergic inflammatory disorder of the airways is selected from the group consisting of asthma, intrinsic or extrinsic asthma bronchiale, acute chronic bronchitis, pulmonary inflammatory reactions secondary to chronic bronchitis, chronic obstructive lung disease, and pulmonary fibrosis.

8. The method of claim 1, wherein the inflammatory disorder of the airways is either idiopathic pulmonary fibrosis or autoimmune lung disease.

9. The method of claim 1, wherein the AMPA receptor modulator is administered in a single or divided dose.

10. A pharmaceutical composition for the treatment of an inflammatory disorder of the airways in a mammal comprising a therapeutically effective amount of an AMPA receptor modulator compound in a pharmaceutically acceptable vehicle.

11. The pharmaceutical composition according to claim 10, comprising from about 0.01 mg to about 100 mg of an AMPA receptor modulator compound per kilogram of patient body weight per dose in a pharmaceutically acceptable oral drug form.

12. The pharmaceutical composition according to claim 10, comprising from about 0.1 mg to about 50 mg of an AMPA receptor modulator compound per kilogram of patient body weight per dose in a pharmaceutically acceptable oral drug form.

13. The pharmaceutical composition according to claim 10, comprising from about 0.01 μg to about 100 μg of an AMPA receptor modulator compound per kilogram of patient body weight per dose in a pharmaceutically acceptable inhalant vehicle.

14. The pharmaceutical composition according to claim 10, comprising from about 0.1 μg to about 50 μg of an AMPA receptor modulator compound per kilogram of patient body weight per dose in a pharmaceutically acceptable inhalant vehicle.

15. The pharmaceutical composition of claim 10, wherein the AMPA receptor modulator is a non-competitive AMPA receptor antagonist.

16. The pharmaceutical composition of claim 15, wherein the non-competitive AMPA receptor antagonist is selected from the group consisting of 1-(4-aminophenyl)-4-methyl-7,8-methylenedioxy-5H-2,3-benzodiazepine (GYKI 52466); 1-(4-aminophenyl)-8-chloro-2-methyl-11H-imidazo[1,2-c][2,3]benzodiazepine (GYKI-47261); (−)-1-(4-aminophenyl)-3-acetyl-4-methyl-7,8-methylenedioxy-3,4-dihydro-5H-2,3-benzodiazepine (GYKI 53773); (−)-1-(4-aminophenyl)-3-methylcarbamoyl-4-methyl-7,8-methylenedioxy-3,4-dihydro-5H-2,3-benzodiazepine (GYKI-53784); 3-(2-chlorophenyl)-2-[2-(3-cyano-pyridin-2-yl)-vinyl]-6-fluoro-3-quinazoline-4-one (CP-526,427); (S)-3-(2-chlorophenyl)-2-[2-(6-diethylaminomethyl-pyridin-2-yl)-vinyl]-6-fluoro-3H-quinazoline-4-one (CP-465,022); 1-(4-amino-phenyl)-3,5-dihydro-4-methyl-3-acetyl-7-methoxy-5H-2,3-benzodiazepine (SYM 2267); N,N-dimethyl-2-[2-(3-phenyl-1,2,4-oxadiazol-5-yl)phenoxy]ethanamine (BIIR 561 CL, irampanel) and their pharmaceutically acceptable acid addition salts.

17. The pharmaceutical composition of claim 10, wherein the AMPA receptor modulator is a competitive AMPA receptor antagonist.

18. The pharmaceutical composition of claim 17, wherein the competitive AMPA receptor antagonist selected from the group consisting of 6-cyano-7-nitroquinoxaline-2,3-dione (CNQ); 6,7-dinitroquinoxaline-2,3-dione PNQ); 2,3-dihydroxy-6-nitro-7-sulfamoyl-benzo(f)quinoxaline NBQX); 1,4,7,8,9,10-hexahydro-9-methyl-6-nitropyrido[3,4-f]quinoxaline-2,3-dione (PNQX, PD 152247); 8-methyl-5-[4-(dimethylsulfamoyl)phenyl]-6,7,8,9-tetrahydro-1H-pyrrolo[3,2-h]isoquinoline-2,3-dione-3-0 (4-hydroxybutiric acid)oxime (NS-1209,SPD-502); 5-[(N-carboxymethyl,N-methyl)amino-methyl]-6-methyl-7-nitro-quinoxaline-2,3-dione; N-((1-(1-carboxymethyl-5,6,7,8-tetrahydro-benzo(f)quinoxaline-2,3-(1H,4H)-dion-9-yl)pyrrol-3-yl)methyl-N′-(4-carboxyphenyl)-urea (LU 115 455); 1,4-dihydro-4-carboxymethyl-6-(1H-imidazol-1-yl)-7-nitro-2,3-quinoxaline-dione (YM 872, zonampanel); 5-[N-(phosphono-methyl)amino-methyl]-7-nitroquinoxaline-2,3-dione (AMP 397A); 1-carboxymethyl-7-(3-carboxy-pyrrol-1-yl)-6-nitroquinoxaline-2,3-(1H,4H)-dione (LU 112 313); 7-chloro-4,5-dihydro-8-(1,2,4-triazol-4-yl)-4-oxo-1,2,4-triazolo[1,5-a]quinoxaline-2-carboxyiic acid (TQX-173); [6,7-dichloro-2(1H)oxoquinoline-3-yl]phosphonic acid (S-17625); 9-carboxymethyl-4,5-dihydro-4-oxo-imidazo[1,2-a]indeno[1,2-e]pyrazin-2-carboxylic acid (Indenone 4f); 2-phosphonoethyl-5-methyl-phenylalanine; 2-amino-3-[(3-carboxymethoxy)-5-methylisoxazol-4-yl]propionate [(S)-AMOA]; (3S,4aR,6R,8aR)-decahydro-6-[2-(1H-tetrazol-5-yl)ethyl]-3-isoquinoline-3-carboxylic acid (LY-293558, LY-326325) and pharmaceutically acceptable salts thereof.
